(19) United States
(12) Patent Application Publication
(10) Pub. No.: US 2019/0320932 A1
(43) Pub. Date: Oct. 24, 2019

(54) STIMULATION PROBE AND STIMULATION APPARATUS

(71) Applicant: NIHON KOHDEN CORPORATION, Tokyo (JP)

(72) Inventors: Shogo SHIRASAKA, Tokorozawa-shi (JP); Norihide SHIMIZU, Tokorozawa-shi (JP); Takanori SATO, Tokorozawa-shi (JP)

(21) Appl. No.: 16/383,794

(22) Filed: Apr. 15, 2019

(30) Foreign Application Priority Data
Apr. 23, 2018 (JP) 2018-082429

(51) Int. Cl. A61B 5/0492

(52) U.S. Cl. CPC A61B 5/0492

(57) ABSTRACT

A stimulation apparatus has a stimulation probe, and a main unit connected to the stimulation probe. The stimulation probe has a housing, an electrode, and a direction adjusting mechanism configured to set a direction of the electrode. The direction adjusting mechanism is configured to selectably fix the electrode in one of a plurality of directions with respect to the housing.

### CROSS-REFERENCE TO RELATED APPLICATION

The present application claims priority to Japanese Patent Application No. 2018-082429 filed on Apr. 23, 2018 the entire content of which is incorporated herein by reference.

### BACKGROUND

The presently disclosed subject matter relates to a stimulation probe and a stimulation apparatus.

In acquiring EMG data by a measuring apparatus, a stimulation probe connected to a main unit of a stimulation apparatus is used. See, e.g., Neuropack X1 by Nihon Kohden (https://us.nihonkohden.com/media/1066/meb-2300-brochure_nmlb-27c-co-02162. pdf). According to a related art stimulation probe, a position of an electrode is fixed.

To apply the electrode to a suitable site, it is sometimes necessary to change a direction of the stimulation probe or a direction of a patient. It adds a burden on the patient when the patient is moved to so as to make the electrode easier to be attached. It is also inconvenient to change the direction of the stimulation probe or the direction of the patient for each examination.

### SUMMARY

Illustrative aspects of presently disclosed subject matter provide a stimulation probe and a stimulation apparatus including the probe, by which a burden on a medical personnel or a patient during an examination can be reduced.

According to an illustrative aspect of the presently disclosed subject matter, the stimulation probe has a housing, an electrode, and a direction adjusting mechanism configured to set a direction of the electrode. The direction adjusting mechanism is configured to selectably fix the electrode in one of a plurality of directions with respect to the housing.

According to an illustrative aspect of the presently disclosed subject matter, the stimulation apparatus has a stimulation probe, and a main unit connected to the stimulation probe. The stimulation probe has a housing, an electrode, and a direction adjusting mechanism configured to set a direction of the electrode. The direction adjusting mechanism is configured to selectably fix the electrode in one of a plurality of directions with respect to the housing.

### BRIEF DESCRIPTION OF DRAWINGS

FIG. 1 is a perspective view of a stimulation apparatus according to an embodiment of the presently disclosed subject matter;

FIGS. 2A to 2C are cross-sectional views taken along the line II-II in FIG. 1, in which FIG. 2A illustrates a state inside a direction adjusting mechanism when a movable switch is in a normal position, FIG. 2B illustrates a state inside the direction adjusting mechanism when the movable switch is moved rearward, and FIG. 2C illustrates a state inside the direction adjusting mechanism when an electrode starts to rotate; and

FIGS. 3A and 3B are side views of a stimulation probe according to the embodiment of the presently disclosed subject matter, in which FIG. 3A illustrates a state in which the electrode rotates upward, and FIG. 39 illustrates a state in which the electrode rotates downward.

### DETAILED DESCRIPTION

Hereinafter, an embodiment of the presently disclosed subject matter is described with reference to the drawings.

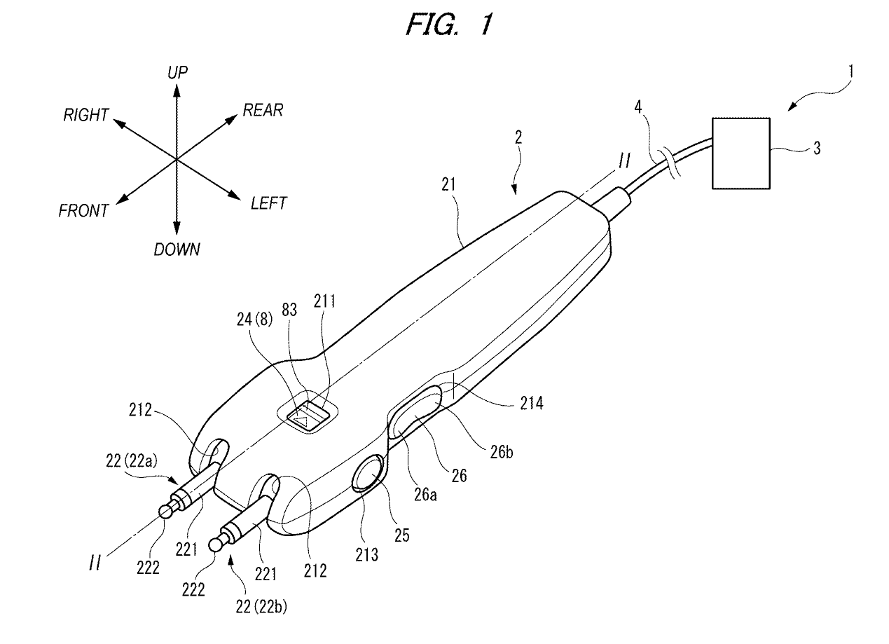

FIG. 1 is a perspective view of a stimulation apparatus 1 according to an embodiment of the presently disclosed subject matter. As illustrated in FIG. 1, the stimulation apparatus 1 includes a stimulation probe 2 and a main unit. The stimulation probe 2 includes a housing 21, an electrode 22, a direction adjusting mechanism 24, a discharge switch 25 for discharging electricity from the electrodes 22 (22a, 22b), and a control switch 26 for changing intensity of the electricity discharged from the electrode 22. The stimulation probe 2 is connected with the main unit via a cord 4. The stimulation probe 2 or the main unit includes a pulse generation device. The pulse generation device generates a pulse current or a pulse voltage. The cord 4 functions as a transmission path of the pulse current or the pulse voltage transmitted from the stimulation probe 2 or the main unit.

In the description, a “left-right direction”, a “front-rear direction”, and a “up-down direction” will be used for the purpose of illustration. These directions are defined with reference to a front direction being a direction of the electrode 22 of the stimulation probe 2. The “up-down direction” includes an “upper direction” and a “lower direction”. The “front-rear direction” includes the “front direction” and a “rear direction”. The “left-right direction” includes a “left direction” and a “right direction”.

The housing 21 has an opening portion 211, an electrode moving slot 212, a first switch hole 213, and a second switch hole 214. The opening portion 211 is provided in the front of each of an upper surface and a lower surface of the housing 21. The upper surface and the lower surface of the housing 21 are symmetrical with respect to a connection surface between the upper surface and the lower surface. Two opening portions 211 are formed at positions that are symmetrical with respect to the connection surface. An operating portion 83 of a movable switch 8, which is a part of the direction adjusting mechanism 24, is exposed from the opening portion 211. The operating portion 83 has a convex shape. The movable switch 8 is movable in a front-rear direction, i.e., the longitudinal direction of the housing 21.

Two electrode moving slots 212 are provided on a front surface of the housing 21. The electrodes 22 (22a, 22b) protrude from the electrode moving slots 212, respectively. The electrode 22 includes an electrode body 221 and a contact portion 222. The electrode body 221 has a cylindrical shape and extends forward. The contact portion 222 is provided at the front side of the electrode body 221. The contact portion 222 has a circular arc shape. The pulse current or the pulse voltage output from the pulse generation device is transmitted to the electrode 22 via the cord 4. The pulse current or the pulse voltage transmitted to the electrode 22 flows to a surface of a subject via the contact portion 222 of the electrode 22. In the present embodiment, the electrodes 22 are paired, in which the electrode 22a is a positive electrode and the electrode 22b is a negative electrode.

The first switch hole 213 is provided on each lateral surface, which is located in the left-right direction of the opening portion 211, of the housing 21. Two first switch holes 213 are disposed in positions that are symmetrical with a center line of the housing 21 in the front-rear direction. The first switch hole 213 is a circular hole. The discharge switch 25 is exposed from the first switch hole 213. The discharge switch 25 has a circular shape. A diameter of the first switch hole 213 is slightly larger than a diameter of the discharge switch 25. When the discharge switch 25 is pressed, a pulse current or a pulse voltage is discharged from the electrode 22.

The second switch hole 214 is provided at the rear side of the first switch hole 213, and is provided on each lateral surface of the housing 21. Two second switch holes 214 are disposed in positions that are symmetrical with the center line of the housing 21 in the front-rear direction. The second switch hole 214 is an elongated hole extending in the front-rear direction, and an end portion thereof has a round shape. The control switch 26 is exposed from the second switch hole 214. A length of an outer circumference of the second switch hole 214 is slightly larger than a length of an outer circumference of the control switch 26. When a front portion 26a of the control switch 26 is pressed, the intensity of electrical stimulation caused by the pulse current or the pulse voltage is increased. Conversely, when a rear portion 26b of the control switch 26 is pressed, the intensity of the electrical stimulation caused by the pulse current or the pulse voltage is weakened.

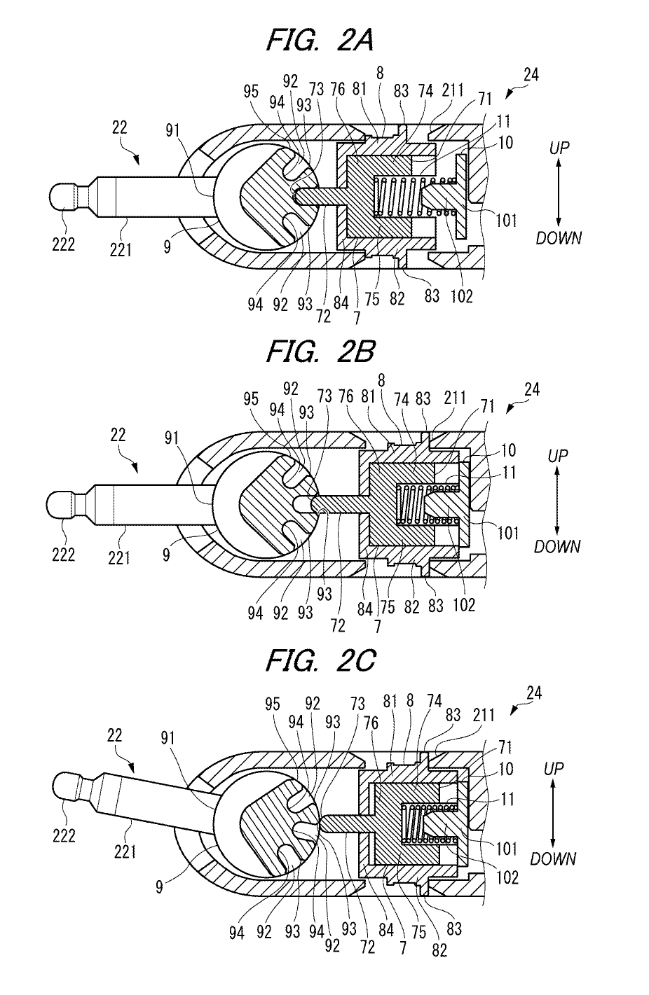

The direction adjusting mechanism 24 is described with reference to FIG. 2. FIG. 2 is a cross-sectional view taken along the line II-II in FIG. 1. FIG. 2A illustrates a state inside the direction adjusting mechanism 24 when a movable switch 8 is in a normal position. FIG. 2B illustrates a state inside the direction adjusting mechanism 24 when the movable switch 8 is moved rearward. FIG. 2C illustrates a state inside the direction adjusting mechanism when an electrode starts to rotate. As illustrated in FIGS. 2A to 2C, the direction adjusting mechanism 24 is provided inside the housing 21. The direction adjusting mechanism 24 includes a direction fixing element 7, a movable switch 8, a selector element 9, a holding base 10, and an elastic member 11.

The direction fixing element 7 includes a base portion 71 and a rod portion 72. The base portion 71 has a U shape when viewed in the II-II cross section. An upper portion 74 and a lower portion 75 of the direction fixing element 7 are both in contact with the movable switch 8 and the elastic member 11. A rod portion 72 is provided substantially at the center of a front portion 76, which is perpendicular to the longitudinal direction of the housing 21 and extends in the up-down direction, of the direction fixing element 7. The rod portion 72 extends forward and is parallel to the longitudinal direction of the housing 21. A rounded portion 73 is formed at a front end of the rod portion 72. The rounded portion 73 has a circular arc shape. The front portion 76 is in contact with the elastic member 11 on a surface opposite to a surface on which the rod portion 72 is provided, that is, on a rear surface.

The movable switch 8 has a substantial U shape when viewed in the II-II cross section. An operating portion 83 is formed on each of an upper portion 81 and a lower portion 82 of the movable switch 8, which are parallel to the longitudinal direction of the housing 21 and extend in the front-rear direction. When two operating portions 83 are provided as shown in the drawing, the thumb or other fingers (e.g., the index finger) can also operate the operating portion. Accordingly, it is preferable that two operating portions 83 are provided. However, two operating portions 83 are not necessarily required, and only one operating portion 83 may be provided. An insertion hole, through which the rod portion 72 of the direction fixing element 7 can be inserted, is formed substantially at the center of a front portion 84 of the movable switch 8. The front portion 84 is perpendicular to the longitudinal direction of the housing 21 and extends in the up-down direction. A width of the insertion hole is slightly larger than the width of the rod portion 72.

A base portion 71 is disposed in space formed by the movable switch 8.

The selector element 9 has a circular shape when viewed in the II-II cross section. An outer circumferential surface 95 is formed on a circumference of the selector element 9. Two receiving portions 91 are formed in the front of the selection portion 9. A rear end of the electrode body 221 can be inserted into the receiving portion 91. When the rear end of the electrode body 221 is inserted into the receiving portion 91, the electrode 22 is integrally connected with the selector element 9.

A selection hole 92 is provided at the rear side of the selector element 9. In the present embodiment, three selection holes 92 are provided in the selector element 9. A front end 94 of the selection hole 92 has a circular arc shape. A rounded portion 93 is formed at the rear end of the selection hole 92. The rounded portion 93 can contact the rounded portion 73 of the rod portion 72.

The holding base 10 has a convex shape when viewed in the II-II cross section. The holding base 10 includes a planar portion 101 and a protruding portion 102. The protruding portion 102 extends forward from the planar portion 101. The protruding portion 102 can be inserted into the elastic member 11. The elastic member 11 is held by inserting the protruding portion 102 into the elastic member 11 and bringing the elastic member 11 into contact with the front portion 76. The elastic member 11 in the present embodiment is a coil spring.

As illustrated in FIG. 2A, the operating portion 83 of the movable switch 8 is generally at a substantially central position (first position) of the opening portion 211. An elastic biasing force of the elastic member 11 acts forward, when the movable switch 8 is in the first position. Accordingly, a forward force is applied to the direction fixing element 7. When the movable switch 8 is in the first position, the front portion 84 of the movable switch 8 is in contact with the front portion 76 of the direction-determining portion 7. Accordingly, the forward force is also applied to the movable switch 8. Therefore, when the movable switch 8 is in the first position, the front portion 84 of the movable switch 8 is positioned at the forefront. At this time, the rod portion 72 is inserted into any one of the selection holes 92, and the front end of the rod portion 72 is positioned in a vicinity of a front end of the selection hole 92.

As shown in FIG. 2B, the operating portion 83 of the movable switch 8 can be moved from the first position to a position (second position) Where the operating portion 83 contacts the rear end of the opening portion 211. When the operating portion 83 is moved rearward, the entire movable switch 8 is also moved rearward. The direction fixing element 7 also moves rearward since the direction fixing element 7 is in contact with the movable switch 8. When the direction fixing element 7 moves rearward, a rearward compressive force is applied to the elastic member 11. Accordingly, the elastic member 11 is compressed. At this time, the rod portion 72 moves to a position (second position) where the rounded portion 73 of the rod portion 72 contacts the rounded portion 93 of the selection hole 92. In the second position, an elastic biasing force against rearward compression acts on the elastic member 11. The rounded portion 73 is in contact with the rounded portion 93 in the second position. Accordingly, the distal end of the rod portion 72 is maintained in contact with a portion of the selector element 9 defining the entrance of the selection hole 92. Therefore, when the movable switch 8 is in the second position, the selector element 9 does not rotate and the electrode 22 does not rotate either without any further operation.

As illustrated in FIG. 2C, the electrode 22 rotates upward when the medical personnel applies an upward force to the electrode 22 based on the state in which the movable switch 8 is in the second position, for example. When the electrode 22 rotates upward, the selector element 9 connected to the electrode 22 rotates. When the selector element 9 rotates, the rounded portion 73 of the rod portion 72 slides over the rounded portion 93 of the selection hole 92 and slides over the rounded portion 93. As a result, the rounded portion 73 contacts the outer circumferential surface 95 of the selector element 9.

When the rounded portion 73 slides over the rounded portion 93, the rod portion 72 moves rearward from the second position. When the rod portion 72 moves rearward, a force is also applied to the direction fixing element 7 in contact with the rod portion 72 in the rear direction. Therefore, a rearward compressive force is applied to the elastic member 11, and thus the elastic member 11 is further compressed. As a result, the direction fixing element 7 is displaced to a third position that is further rearward from the second position. At this time, the movable switch 8 does not move rearward. That is, the position of the movable switch 8 remains unchanged even when the direction fixing element 7 is displaced to the third position.

When the medical personnel applies a force to the electrode 22 to rotate the selector element 9 based on the state in which the direction fixing element 7 is in the third position, the rounded portion 73 of the rod portion 72 contacts the rounded portion 93 of another selection hole 92 that is different from the selection hole 92 inserted by the rounded portion 73 in the first position. At this time, the elastic biasing force of the elastic member 11 acting forward is also applied to the direction fixing element 7. Therefore, the rod portion 72 is inserted into the other selection hole 92 when the rounded portion 73 of the rod portion 72 moves and slides over the rounded portion 93 of the other selection hole 92, and moves a predetermined distance. The direction fixing element 7 returns to the first position together with the movable switch 8.

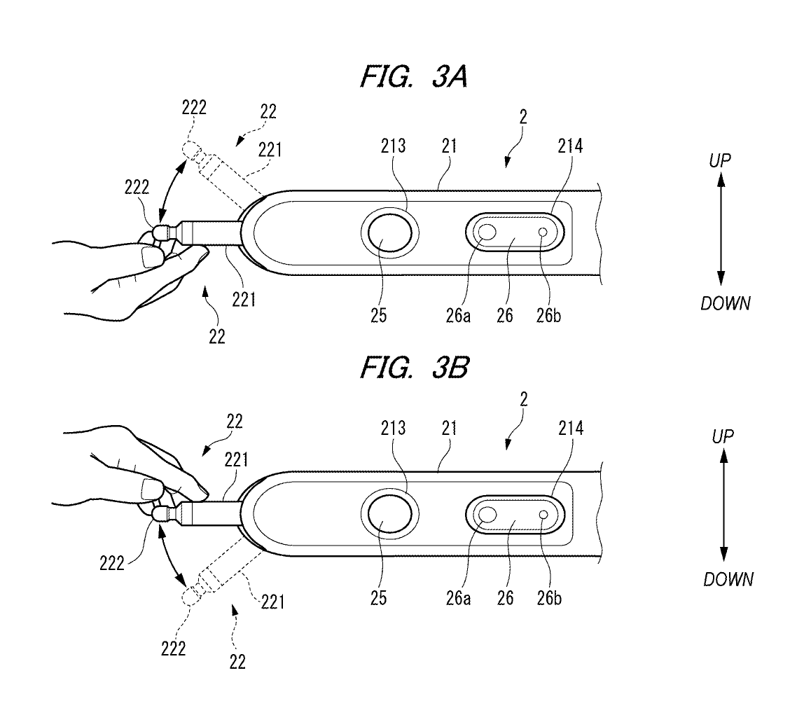

FIGS. 3A and 3B are side views of the stimulation probe 2. FIG. 3A illustrates a state in which the electrode 22 rotates upward, and FIG. 3B illustrates a state in which the electrode 22 rotates downward.

As illustrated in FIG. 3A, the electrode 22 rotates upward when the medical personnel moves the operating portion 83 of the movable switch 8 rearward and pushes the electrode 22 upward.

As illustrated in FIG. 3B, the electrode 22 rotates downward when the medical personnel moves the operating portion 83 of the movable switch 8 rearward and presses the electrode 22 downward. The electrode 22 is rotated from about 30° to 60° in the up-down direction with reference to a position of the electrode 22 in a normal state. A rotation angle of the electrode 22 is not limited to about 30° to 60°, and may be rotated to about 90°, for example.

According to the stimulation probe 2 of the above configuration, the electrode 22 can be fixed in a plurality of directions, so that the electrode 22 can be applied to a suitable place without changing the direction of the housing 21 of the stimulation probe 2 and the direction of the patient.

As described above, according to the stimulation probe 2 of the above configuration, the burden on the medical personnel or the patient during the examination can be reduced.

According to the stimulation probe 2 of the above configuration, when the medical personnel moves the movable switch 8 and the electrode 22, the selector element 9 moves, which is integrally installed together with the electrode 22. When the movable switch 8 returns to an original position thereof (first position), the rod portion 72 provided in the direction fixing element 7 is inserted into any one of the selection holes 92. Accordingly, the direction of the electrode 22 is set Therefore, the medical personnel can apply the electrode 22 to a suitable place without changing the direction of the stimulation probe main body and the direction of the patient.

As described above, according to the stimulation probe 2 of the above configuration, the burden on the medical personnel or the patient during the examination can be reduced.

According to the stimulation probe 2 of the above configuration, the direction of the electrode 22 can be set by simply rotating the selector element 9.

According to the stimulation probe 2 of the above configuration, the state in which the rod portion 72 is inserted into the selection hole 92 is maintained by the elastic biasing force, toward the direction of the selector element 9, of the elastic member 11, unless the movable switch 8 is moved by the medical personnel.

According to the stimulation probe 2 of the above configuration, the direction of the electrode 22 is also maintained since the distal end of the rod portion 72 is maintained in contact with the portion of the selector element 9 defining the entrance of the selection hole

According to the stimulation probe 2 of the above configuration, the direction fixing element 7 is displaced to the second position when the medical personnel moves the movable switch 8 rearward. In the second position, the selector element 9 does not rotate since the rounded portion 73 formed on the distal end of the rod portion 72 is in contact with the rounded portion 93 formed on the portion of the selector element 9 defining the entrance of of the selection hole 92. Further, when the medical personnel applies rotary power to the selector element 9, the distal end of the direction fixing element 7 slides over the rounded portion 93 of the selector element 9 and contacts the outer circumferential surface 95 of the selector element 9. Accordingly, the direction fixing element 7 is displaced to the third position. In the third position, the rod portion 72 is inserted into another selection hole 92 by further rotating the selector element 9.

According to the stimulation probe 2, of the above configuration, the electrode 22 can be fixed in the same direction as the longitudinal direction of the housing 21 or in a direction vertically inclined with respect to the longitudinal direction of the housing 21. Therefore, the electrode 22 can be applied to a suitable place regardless of the direction of the patient.

Further, according to stimulation probe 2 of the above configuration, the operating portions 83 are provided on the upper and lower surfaces of the housing 21, so that the medical personnel can operate the operating portion 83 regardless of the direction of the stimulation probe 2 when the medical personnel picks up the stimulation probe 2. Therefore, an easily operable state can be maintained without being influenced by a dominant hand or the operation state of the medical personnel. Further, the same operability can be realized even when the upper and lower surfaces are changed since the upper and lower surfaces of the housing 21 are plane-symmetrical.

According to the stimulation apparatus 1 of the above configuration, the electrode 22 can be fixed in a plurality of directions, so that the electrode 22 can be applied to a suitable place without changing the direction of the stimulation probe 2 connected to the main unit and the direction of the patient.

As described above, according to the stimulation apparatus 1 of the above configuration, the burden on the medical personnel or the patient during the examination can be reduced.

In the example described above, the electrode 22 is fitted into the selector element 9, and the selector element 9 is rotated by rotation of the electrode 22. However, this is a non-limiting example. For example, an insertion hole for inserting the electrode 22 may be provided in the selector element 9, and the direction of the electrode 22 may be changed by inserting the electrode 22 in various directions into the insertion hole,

In the example described above, three selection holes 92 are provided in the selector element 9. However, this is a non-limiting example. Two or more selection holes 92 may be provided in the selector element 9.

The stimulation probe 2 is connected with the main unit via the cord 4 in the present embodiment, but the presently disclosed subject matter is not limited to this example. For example, the stimulation probe 2 may be connected with the main unit by wireless communication.

In the example described above, the movable switch 8 is movable in the front-rear direction, i.e., the longitudinal direction of the housing 21. However, this is a non-limiting example. The movable switch 8 may be, for example, a switch that is movable in the left-right direction, which is the direction of the short side of the housing 21, or may be a switch to be pressed. In this case, when the movable switch 8 is moved or pressed, the direction fixing element 7 may move in the front-rear direction by a mechanical element that converts a direction of a force.

While the presently disclosed subject matter has been described with reference to certain embodiments thereof, the scope of the presently disclosed subject matter is not limited to the embodiments described above, and it will be understood by those skilled in the art that various changes and modifications may be made therein without departing from the scope as defined by the appended claims.

What is claimed is:

1. A stimulation probe comprising:
a housing;
an electrode; and
a direction adjusting mechanism configured to set a direction of the electrode,
wherein the direction adjusting mechanism is configured to selectably fix the electrode in one of a plurality of directions with respect to the housing.

2. The stimulation probe according to claim 1, wherein the direction adjusting mechanism comprises:
a selector element connected to the electrode, the selector element comprising at least two selection holes configured to select a direction of the electrode;
a movable switch provided in the housing; and
a direction fixing element configured to move in accordance with a movement of the movable switch, the direction fixing element comprising a rod portion configured to be inserted into one of the selection holes and, and
wherein the rod portion is configured to fix the direction of the electrode by being inserted into one of the selection holes.

3. The stimulation probe according to claim 2, wherein the selector element is rotatable with respect to the housing.

4. The stimulation probe according to claim 3, wherein the direction adjusting mechanism further comprises an elastic member and a holding base configured to hold the elastic member,
wherein the elastic member is arranged to contact with direction fixing element and the holding base, and
wherein the elastic member is arranged to be compressed in a direction toward the holding base when the movable switch is moved to move the direction fixing element.

5. The stimulation probe according to claim 4, wherein the elastic member is configured to apply a forwardly biasing force to the direction fixing element in reaction to being compressed, and
wherein the elastic biasing force maintains a distal end of the rod portion to be in contact with a portion of the selector element defining an entrance of one of the selection holes.

6. The stimulation probe according to claim 2, wherein the rod portion comprises a first nonangular rounded portion at a distal end of the rod portion,
wherein the selector element comprises a second nonangular rounded portion at a portion of the selector element defining an entrance of one of the selection holes,
wherein the direction fixing element is movable to
a first position at which the movable switch is not moved,
a second position at which the movable switch is moved rearward, and
a third position rearward from the second position, and
wherein the first nonangular rounded portion and the second nonangular rounded portion are in contact with each other when the direction fixing element is in the second position, and when the selector element rotates, the distal end of the rod portion of the direction fixing element is brought into contact with an outer circumference of the selector element such that the direction fixing element is moved to the third position.

7. The stimulation probe according to claim 1, wherein the plurality of directions comprises a longitudinal direction of the housing and a direction vertically with respect to the longitudinal direction of the housing.

8. The stimulation probe according to claim 2, wherein the housing comprises an opening portion at upper and lower surface of the housing to expose an operating portion of the movable switch.

9. The stimulation probe according to claim 8, wherein the upper and lower surfaces of the housing are plane-symmetrical.

10. A stimulation apparatus comprising:
a stimulation probe; and
a main unit connected to the stimulation probe,
wherein the stimulation probe comprises:
a housing;
an electrode; and
a direction adjusting mechanism configured to set a direction of the electrode, and
wherein the direction adjusting mechanism is configured to selectably fix the electrode in one of a plurality of directions with respect to the housing.
